(19) United States
(12) Patent Application Publication
(10) Pub. No.: US 2023/0000742 A1
(43) Pub. Date: Jan. 5, 2023

(54) HAIR-STRAIGHTENING COMPOSITIONS

(71) Applicant: BEAUTY & BUSINESS S.p.A., Milano (IT)

(72) Inventors: Giovanni Villa, Cornate d'Adda (MB) (IT); Andrea Bielli, Caravaggio (BG) (IT); Emanuela Facchetti, Romano di Lombardia (BG) (IT)

(73) Assignee: BEAUTY & BUSINESS S.p.A., Milano (IT)

(21) Appl. No.: 17/808,305

(22) Filed: Jun. 23, 2022

(30) Foreign Application Priority Data
Jun. 25, 2021 (IT) 102021000016733

(51) Int. Cl. A61K 8/365; A61K 8/36; A61Q 5/04; A61K 8/41; A61K 8/06

(52) U.S. Cl. CPC A61K 8/365; A61K 8/36; A61Q 5/04; A61K 8/416; A61K 8/062

(57) ABSTRACT

Disclosed are hair-straightening compositions in liquid form with an aqueous base, or in the form of an emulsion with a fatty phase, comprising glyoxylic acid and levulinic acid.

This non-provisional application claims priority to and the benefit of Italian Patent Application no. No. 102021000016733 filed Jun. 25, 2021, the content of which is incorporated herein by reference in its entirety.

The subject of the invention is hair-straightening compositions in liquid form with an aqueous base, or in the form of an emulsion with a fatty phase, comprising glyoxylic acid and levulinic acid.

### PRIOR ART

In the field of cosmetic products for the hair treatment, straightening agents, which are able to vary the shape of the hair from curly/wavy to straight, occupy substantial shares of the market.

One of the major problems with hair-straightening compounds is that they are highly aggressive towards the hair.

The disulphide bonds (—S—S—) of keratin fibre help to maintain the natural smooth or curly configuration of the hair. Said bonds are formed by oxidation of the thiol groups of two cysteines, with the formation of a cystine molecule. The remodelling of said bonds permanently modifies the shape of the hair. The hair structure can also be modified semi-permanently, by using chemical agents able to bond covalently to the keratin fibres.

Four general methods are currently used to reshape the hair:

1: Permanent Straightening With Reducing Agents

This method involves reduction of cystine to cysteine, followed by reconfiguration of the hair fibres and re-formation of cystine by oxidation. Thiol compounds such as ammonium thioglycolate are used as reducing agents. Hydrogen peroxide is generally used for the oxidation stage. One of the drawbacks of reducing agents is that they weaken the hair structure. The reasons are: 1) formation of lanthionine during the reduction stage because of the alkaline pH; 2) formation of cysteic acid during the oxidation stage; 3) reduction of tensile properties due to transverse swelling and longitudinal contraction effects on the hair during treatment.

2: Permanent Straightening With Alkaline Agents

This method involves the use of strong bases, generally alkaline earth metal hydroxides, to modify the disulphide bridges (—S—S—) irreversibly with the formation of lanthionine (—CH₂—S—CH₂—) and lysinoalanine. Unlike the disulphide bridge, responsible for the tenacity of the hair, the lanthionine bond considerably weakens the hair structure. The irreversible transformation of the cystine residues makes it impossible to perform a subsequent treatment to change the shape of the hair again.

The most commonly used base is sodium hydroxide, in compositions at pH 12-14, a very aggressive treatment for both the scalp and the hair, which are irreparably damaged. In addition to lanthionine formation, hydrolysis of the peptide and isopeptide bonds may take place. There is also massive loss of the lipid component of the cuticle. At the end of treatment, the hair is extremely dry and fragile, and the use of leave-on conditioning products is required to improve combability, reduce the static effect and protect the hair cuticle.

3: Semi-Permanent Straightening Based on Formaldehyde

Semi-permanent hair straightening, also known as Brazilian straightening, is a method for straightening the hair effectively with the combined use of a formaldehyde-based product and a hair iron. Formaldehyde (or compounds that release it) creates new bonds in the polypeptide structure, forming inter-chain crosslinks and enabling the straightening induced by the iron to be maintained. The typical concentration of formaldehyde used in such types of straightener ranges from 5 to 10% w/w. The straightening process is promoted by removal of water during the drying stage, and by the heat supplied by the iron. Due to the volatility of the molecule, a considerable amount of formaldehyde evaporates during the treatment. The use of formaldehyde has been regulated, and its maximum concentration was set at 0.2% v/v because of its strong irritant potential towards the eyes and respiratory mucosa, and its classification as a carcinogen in 2004. Formaldehyde-based straighteners can therefore no longer be used.

4: Semi-Permanent Straightening With Carboxylic Acids

To obviate all the drawbacks of the methods described above, the use of carboxylic acids of formula RCOCOOH (wherein R═H, alkyl), especially glyoxylic acid and/or pyruvic acid, has been proposed.

The action mechanism of glyoxylic acid (similar to that of formaldehyde) and the structure-activity correlations were described by Boga C. et al. in “International Journal of Cosmetic Science, 2014, 36, 459-470.

To perform a straightening effect, the amount of glyoxylic acid present in the composition must be at least 5% w/w, but effective, lasting straightening is only obtained at concentrations of 10-20% w/w of the total weight of the composition. The straightening performance sometimes fails to meet expectations. In particular, the combability of the hair after treatment is often unsatisfactory and the hair remains rather frizzy, especially in the case of level 4 curly hair (“very curly” on the scale devised by De La Mettrie et al., Hum. Biol. 2007, 79, 265-281).

It is important to note that treatment with a semi-permanent straightener at a highly acid pH gives rise to a colour change and/or colour fading in both natural hair and already dyed hair. This problem sometimes leads to dissatisfaction, and the need to perform a supplementary tonalising treatment.

WO2015094788 describes a method designed to give form to a fibrous material like hair, using an active agent, a photocatalyst and electromagnetic radiation. Levulinic acid is listed among the active agents.

WO2013092959 describes a hair-straightening treatment involving the use of a) one or more liquid fatty substances, b) one or more surfactants, and c) one or more dicarbonyl compounds other than a). The preferred dicarbonyl compounds are alpha-keto acids such as glyoxylic acid and pyruvic acid. Levulinic acid is also mentioned, but no example of a composition containing it is provided. Ingredients a) and b) are contained in an inverse emulsion (water-in-oil), while ingredient c) is in a separate composition. The fatty phase contained in the straightening compositions in emulsion tends to produce a considerable amount of smoke during the passage of the hot iron, so that hair needs to be rinsed before ironing, leading to a reduction in straightening performance.

JP2019123701 describes a hair treatment composition at pH 1.5-3 containing 13-30 mass% of levulinic acid and 1-2 mass % of glyoxylic acid, so that the total content of levulinic acid and glyoxylic acid is at least 15%. According to the inventor, the small percentage of glyoxylic acid prevents fading of the cosmetic colour present on the hair, and the composition provides sufficient straightening. However, the patent does not mention the duration of said straightening effect following subsequent washes, and there is no mention of an efficient, persistent/semi-permanent straightening effect.

The present invention aims to develop a straightening composition that ensures efficient, lasting hair straightening. The invention also aims to reduce frizziness to a lasting extent, even on the most difficult curls, and to make the hair not only more manageable during treatment, but also easily combed and soft after treatment.

### DESCRIPTION OF THE INVENTION

It has surprisingly been found that by combining glyoxylic acid with levulinic acid in suitable ratios, a straightening effect is obtained which is better and more lasting than that obtainable with the straighteners on the market which only contain glyoxylic acid. An improvement is also obtained in terms of combability and softness, with a reduction in frizziness even after several washes.

### BRIEF DESCRIPTION OF THE FIGURES

FIG. 1 shows two representative images showing the photos after processing.

FIG. 2 shows that at time zero, formula B improves frizziness by nearly 10% compared to reference formula E.

FIG. 3 shows frizzy hair effect after washing.

FIG. 4 shows that at the end of straightening of the locks, formula A exhibited better compatibility on dry and on wet hair.

FIG. 5 shows that at the end of straightening of the locks, formula B exhibited better compatibility on dry and on wet hair.

FIG. 6 shows a representative example of the straightening efficacy.

FIG. 7 shows the straightening power of the formulas containing levulinic acid.

FIG. 8 shows locks representative of each experimental group.

A first subject of the invention therefore relates to semi-permanent hair straightening compositions in liquid form with an aqueous base, or in the form of an emulsion with a fatty phase, comprising 15 to 20% by weight of glyoxylic acid and 0.1 to 5% by weight of levulinic acid.

“Semi-permanent straightening composition” here means a composition able to maintain the straightening effect for up to three months (Boga C et al., “Formaldehyde replacement with glyoxylic acid in semi-permanent hair straightening: a new and multidisciplinary investigation” in International Journal of Cosmetic Science, 2014, 36, 459-470).

The compositions according to the invention, in liquid form with an aqueous base or in the form of an emulsion with a fatty phase (fatty alcohols, fatty acids and the esters thereof), not exceeding 15% by weight (unless otherwise specified, in the present description the percentages by weight are percentages in relation to the total weight of the composition), comprise glyoxylic acid and levulinic acid.

Said compositions give rise to an efficient, persistent straightening effect, and also reduce frizziness. The compositions according to the invention are advantageous for professional users as from the application stage of the treatment, ensuring the hair is manageable and the straightening iron glides easily. The compositions according to the invention leave the hair feeling soft to the touch and easy to comb, maintain excellent straightening results over time and minimise frizziness, even after several washes. The application method does not involve exposure to electromagnetic radiation. The compositions according to the invention do not necessarily need rinsing and produce very little smoke during the passage of the hot iron.

In the compositions according to the invention, levulinic acid is preferably present in percentages by weight ranging from 2% to 3%. The ratio between levulinic acid and glyoxylic acid ranges between 0.004% and 1%, preferably between 0.1% and 0.2%.

The pH of the composition can range between 0.5 and 3.0, preferably between 0.8 and 2.0.

The compositions according to the invention can also contain other ingredients such as pyruvic acid and non-ionic, amphoteric or cationic surfactants.

Examples of non-ionic surfactants which can be used are ethoxylates (polysorbate-20 and other polysorbates; PEG-40 hydrogenated castor oil, PEG-7 glyceryl cocoate and other PEG lipids; poloxamer 407, poloxamer 187 and other poloxamers), alkyl glycosides (coco glucoside, decyl glucoside, lauryl glucoside), alkanolamides (cocamide MEA, cocamide DEA, cocamide MIPA) and amino oxides (lauramine oxide, cocamine oxide, cocamidopropylamine oxide).

Amphoteric surfactants comprise, for example, alkyl betaine (coco-betaine), alkylamidopropyl betaine (cocamidopropyl betaine, lauramidopropyl betaine, hydroxysultaines (cocamidopropyl hydroxysultaine, lauramidopropyl hydroxysultaine), amphoacetates (sodium cocoamphoacetate) and amphodiacetates (disodium cocoamphodiacetate).

Cationic surfactants comprise, for example, quaternary ammonium salts such as chloride, bromide, methylsulphate and (C10-C24)-alkyltrimethylammonium ethylsulphate, preferably chloride, bromide, methylsulphate and (C16-C22)-alkyltrimethylammonium ethylsulphate. Some examples reported with their INCI names comprise cetrimonium chloride, steartrimonium chloride, behentrimonium chloride, and behentrimonium methosulphate. Other cationic surfactants comprise quaternary ammonium salts such as chloride, bromide, methylsulphate and (C10-C24) -dialkyldimethylammonium ethyl sulphate, preferably chloride, bromide, methylsulphate and (C16-C22)-dialkyldimethylammonium ethyl sulphate. Some examples reported with their INCI names comprise dicetyldimonium chloride and distearyldimonium chloride. Other cationic surfactants comprise quaternary ammonium salts such as (hexadecylamidopropyl)-trimethylammonium chloride. Palmitamidopropyltrimonium chloride.

The percentages of said surfactants in the compositions according to the invention range from 0.01% to 10% by weight, preferably from about 0.1% to 5%.

The compositions can also contain cationic viscosity-controlling agents or mixtures thereof in percentages by weight ranging from 0.01% to 5%, preferably from about 0.1% to 3%.

Examples of cationic viscosity-controlling agents include hydroxypropyl guar derivatives, commonly identifiable by their INCI name Guar Hydroxypropyltrimonium Chloride and available on the market in different grades under the tradename Activsoft; Dehyquart® Guar; Ecopol; EcoSmooth™; Guarquat; iQUAT GUAR; Jaguar®; N-Hance; Polycos. Said class also includes the hydroxypropyl derivatives of hydroxypropyltrimonium guar hydrochloride. Of these, the polymer identified by the INCI name Hydroxypropyl Guar Hydroxypropyltrimonium Chloride and available on the market in various grades under the tradename N-Hance™; Polycos; Polycare® Split Therapy; iQUAT GUAR; Jaguar®; Guarquat is preferred. Other examples are quaternium-18 bentonite, quaternium-18/benzalkonium bentonite, quaternium M-18 hectorite, TEA-hydrochloride.

The compositions according to the invention can also include solvents, fats, polymers, oligosaccharides and modified oligosaccharides, carbohydrates and derivatives, glycerols, polyols and derivatives, triglycerides, hydrocarbons, lanolin and derivatives, opacifiers, silicones, protein hydrolysates, amino acids, complexing agents, UV filters, pigments, preservatives and fragrances.

Water-soluble organic solvents include, for example, glycerols, glycols, glycol ethers and polyols containing 2 to 6 carbon atoms. The glycols are ethylene glycol, propanediol, butanediols and pentanediols. Polyalkyl glycols are, for example, polyethylene glycols, polypropylene glycols, and related products to which ethylene oxide with a molecular weight of up to 1000 D is added. Up to 30% by weight of the organic solvent can be present.

The compositions according to the invention can contain cationic polymer conditioners such as quaternised hydroxyethyl cellulose derivatives (Polyquaternium-4, Polyquaternium-10 and Polyquaternium-24); acrylamide and diallyl dimethylammonium chloride copolymers (Polyquaternium-7); acrylic acid and diallyl dimethylammonium chloride copolymers (Polyquaternium-22); poly-diallyl dimethylammonium chloride (Polyquaternium-6).

A particular cationic polymer is the crosslinked homopolymer of methacryloylethyl trimethylammonium chloride (Polyquaternium-37). Other cationic polymers can be Polyquaternium-2; Polyquaternium-4; Polyquaternium-5; Polyquaternium-11; Polyquaternium-16; Polyquaternium-28; Polyquaternium-39; Polyquaternium-43; Polyquaternium-44; Polyquaternium-47; Polyquaternium-51; Polyquaternium-53; Polyquaternium-55; Polyquaternium-67; Polyquaternium-68; Polyquaternium-69; Polyquaternium-71; Polyquaternium-74; Polyquaternium-77; Polyquaternium-78; Polyquaternium-80; Polyquaternium-81; Polyquaternium-88.

The cationic polymers present in the composition can range from 0.01% to 5% by weight, preferably from about 0.1% to 3%.

The compositions can also contain silicones such as cyclosilicones (Cyclomethicone, Cyclopentasiloxane, Cyclohexasiloxane); methyl trimethicones (Methyltrimethoxysilane); polydimethyloxanes (Dimethicone); polymethylsiloxanes (Methicone); polyphenylmethylsiloxanes (Phenyl Trimethicone); silicone rubbers (Dimethiconol) and mixtures thereof (Cyclopentasiloxane (and) Dimethiconol, Dimethicone (and) Dimethiconol); silicone waxes (Cetyl Dimethicone); Amodimethicones (Amodimethicone). They can also contain dimethicone copolyols such as Bis-PEG/PPG-20/20 Dimethicone (ABIL® B 8832); Cetyl PEG/PPG-10/1 Dimethicone (ABIL® EM 90); PEG/PPG-14/4 Dimethicone (ABIL® B 8851); DOWSIL™ ES-5612. They can also contain silicone surfactants such as PEG-10 Dimethicone and PEG-12 Dimethicone, and cationic silicones such as Quaternium-80 (ABIL® Quat 3272) and Silicone Quaternium-22 (ABIL® T QUAT 60). The silicones are preferably present in amounts ranging from 0.01% to 5% by weight, more preferably from 0.1% to 5%.

The compositions according to the invention can also contain vegetable oils such as almond oil, argan oil, avocado oil, castor oil, sesame oil, olive oil, jojoba oil, babassu oil, shea butter, linseed oil and sunflower oil. The natural oils are preferably present in an amount ranging from 0.001% to 1% by weight.

The compositions can also contain opacifiers selected from ammonium styrene/acrylates copolymers, DEA-styrene/acrylates/DVB copolymer, guanine, mica, styrene/acrylamide copolymer, styrene/acrylates copolymer, and styrene/DVB copolymer; opacifiers having rheological corrector properties such as behenamide, erucamide, Nylon-12, Nylon-66, oleamide, oleyl palmitamide, stearamide, stearamide DEA-distearate, stearamide DIBA-distearate and stearyl erucamide. Said ingredients are present in an amount ranging from 0.01% to 3% by weight, preferably from 0.1% to 2%.

The compositions according to the invention can also contain protein hydrolysates of animal origin, such as hydrolysed elastin, collagen, keratin, silk or milk proteins. They can optionally be in salt form and mixed with other ingredients. They are available on the market in different grades, for example under the tradenames Crotein™; Kereffect™; Nutrilan®; Promois; SpecPure®; Vari®Ker; Vari®Silk.

The proteins or protein hydrolysates can also be of plant origin, such as proteins or protein hydrolysates obtained from soy, almonds, peas, potatoes, linseed, corn and wheat, available on the market, for example, under the name Crodasone™; Fision®; Gluadin®; Granosol®; Keraveg™; Kereffect™; Natpure®; Promois; Vari®Rice

The hydrolysates can also be quaternised, such as Keravis™ PE (Aqua (and) Hydrolyzed Vegetable Protein PG-Propyl Silanetriol) and Kerestore™ 2.0 (Laurdimonium Hydroxypropyl Hydrolyzed Keratin). The protein hydrolysates or derivatives thereof can be present in amounts ranging from 0.1 to 10% by weight, preferably from 0.1 to 2.5%.

The compositions can also contain amino acids selected from glycine, sarcosine, lysine, serine, glucosamine, glutamic acid, carnitine, acetylcarnitine, alanine, arginine, asparagine, aspartic acid, cysteine, glutamine, histidine, isoleucine, leucine, lysine, methionine, phenylalanine, proline, serine, threonine, tryptophan, tyrosine and valine, preferably arginine, asparagine, glutamine, histidine, lysine, proline and tryptophan.

The amino acids can be present in amounts ranging from 0.01 to 10% by weight, preferably from 0.01 to 3%.

The compositions can also include a direct non-ionic, cationic or anionic hair dye.

Examples of non-ionic dyes include 2-amino-6-chloro-4-nitrophenol, 4-nitro-o-phenylenediamine, 4-amino-3 -nitrophenol, 4-[(2-hydroxyethyl)amino]-3-nitrophenol (Jarocol NHEAP®); 4-[(3-hydroxypropyl)amino]-3-nitrophenol (Jarocol Red BN®), 1-(2-hydroxyethoxy)-2-[(2-hydroxyethyl)amino]-5-nitrobenzene (HC Yellow No. 4) and 1-[(2-hydroxyethyl)amino]-2-nitrobenzene (HC Yellow No. 2HC Red No. 1, HC Red No. 3, HC Red No. 13, Disperse violet 1, HC Yellow No. 13, Disperse Red 17, Disperse blue 377.

Examples of cationic dyes include Basic Yellow 57, Basic Yellow 87, Basic yellow 40, Basic violet 16, Basic violet 2, Basic Brown 17, Basic Orange 31, Basic Red 51, Basic Red 76, HC Blue 15, HC Blue 16, Hc blue 17, Basic blue 75, Basic brown 16, Basic blue 124.

Examples of anionic dyes include Acid Yellow 1, Acid Yellow 3, Acid Yellow 23, Acid Orange 7, Acid Red 33, Acid Red 40, Acid Red 52, Acid Red 92, Acid Violet 43, Acid Blue 62, Acid Blue 9, Acid blue 7, Acid black 1, tetrabromophenol blue.

The dyes can be contained in the composition alone or in mixtures, in amounts ranging from about 0.01 to 4.0% by weight.

The composition can also include natural direct dyes, such as dyes based on lawsone, juglone, alizarine, purpurine, carminic acid, kermesic acid, purpurogallin, protocatechualdehyde, indigo, isatin, curcumin, spinulosine and apigenidine. Extracts or decoctions containing said natural dyes can also be used.

For further typical dye compounds which can be used in the composition, reference can be made to the “Dermatology” series, edited by Ch. Culnan and H. Maibach, Verlag Marcel Dekker Inc., New York, Basel, 1986, volume 7, Ch. Zviak; “The Science of Hair Care”, chapter 7, pp. 248-250 (substantive dyes), F. Mearelli “Le piante tintorie nella colorazione dei capelli”, and “European Inventory of Cosmetic Raw Materials”, published by the European Union, obtainable from Bundesverband Deutscher Industrie-und Handelsunternehmen fur Arzneimittel, Reformwaren and Körperpflegemittele. V., Mannh.

Pigments such as iron oxides, titanium oxides, zinc oxides, chromium oxides, ultramarine, manganese violet and ferric ferrocyanide can also be used. Other pigments which can be used are those marketed under the names Watersperse® (S.A Color); Unipure (Sensient); Cellini® (BASF); Distinctive® (Re source of Nature), Colorona® (Merck), WD (Daito Kasei).

The pigments can be included in amounts ranging from 0.01 to 10% by weight of the total weight, preferably from 3% to 8%.

The compositions can also include preservatives and fragrances in amounts ranging from 0.01% to 2% by weight and pH adjusters in amounts ranging from 0.01% to 10%, preferably from 0.01% to 2% by weight.

A further subject of the invention relates to a semi-permanent hair straightening method which comprises application to the hair of the compositions according to the invention and subsequent heat treatments, washing and rinsing.

In further detail, the method according to the invention can be implemented, for example, according to two alternative procedures, shown in the schemes below:

Application Method No. 1:
Wash hair twice with a deep-cleansing shampoo. Proceed with total drying with a hairdryer.
Distribute product according to the invention on hair, and leave to act for 20 min. at room temperature.
Without rinsing, brush hair and pass straightening iron at 200° 6 to 15 times, depending on hair type.
apply a rehydrating mask to the damp hair, leave to act for 5 min. and rinse. Dry with hairdryer.

Application Method No. 2:
Wash hair twice with a deep-cleansing shampoo. Proceed with total drying with a hairdryer.
Distribute product according to the invention on hair, and leave to act for 20 min. under a heat source.
Apply a rehydrating mask to the damp hair, leave to act for 5 min. and rinse quickly.
Dry hair up to 80% with a hairdryer, then brush and pass straightening iron at 200° 6 to 15 times, depending on hair type.
Rinse until product has been totally washed away.

The following examples further illustrate the invention. The percentages indicated are always expressed by weight in relation to the total weight of the composition.

Table 1 illustrates formulas A, B, C according to the invention and reference Formula E. Formulas G and H, with a levulinic acid glyoxylic acid content as described in JP2019123701, are also present. Said formulas are lotions not containing a fatty phase.

TABLE 1

| | | | | E | | |
|---|---|---|---|---|---|---|
| Ingredients | A | B | c | (ref.) | G | H |
| (INCI) | % | % | % | % | % | % |
| AQUA | q.s. to | q.s. to | q.s. to | q.s. to | q.s. to | q.s. to |
| (WATER) | 100 | 100 | 100 | 100 | 100 | 100 |
| GLYOXYLIC | 17.5 | 17.5 | 17.5 | 17.5 | 0 | 2 |
| ACID | | | | | | |
| PROPYLENE | 3.0 | 3.0 | 3.0 | 3.0 | 3.0 | 3.0 |
| GLYCOL | | | | | | |
| LEVULINIC | 1.0 | 2.5 | 5.0 | — | 30.0 | 30.0 |
| ACID | | | | | | |
| CETRIMONIUM | 0.75 | 0.75 | 0.75 | 0.75 | 0.75 | 0.75 |
| CHLORIDE | | | | | | |
| BIS-PEG/ | 0.5 | 0.5 | 0.5 | 0.5 | 0.5 | 0.5 |
| PPG-20/20 | | | | | | |
| DIMETHICONE | | | | | | |
| POLY- | 0.2 | 0.2 | 0.2 | 0.2 | 0.2 | 0.2 |
| SORBATE-20 | | | | | | |
| Active ingredients | ≤1 | ≤1 | ≤1 | ≤1 | ≤1 | ≤1 |

Table 2 shows formula D according to the invention and reference formula F. Said formulas are emulsions containing a fatty phase.

TABLE 2

| | D | F (ref.) |
|---|---|---|
| Ingredients (INCI) | % | % |
| AQUA (WATER) | q.s. to | q.s. to |
| | 100 | 100 |
| PARAFFINUM LIQUIDUM (MINERAL OIL) | — | 20 |
| GLYOXYLIC ACID | 17.5 | 17.5 |
| PETROLATUM | — | 8.0 |
| CETEARYL ALCOHOL | 10.8 | 6.0 |
| STEARYL ALCOHOL | — | 4.0 |
| CETEARETH-50 | — | 3.0 |
| CETEARETH-25 | — | 2.0 |
| LEVULINIC ACID | 2.5 | — |
| CETRIMONIUM CHLORIDE | 2.5 | — |
| BEHENTRIMONIUM METHOSULPHATE | 0.9 | — |
| QUATERNIUM-87 | 0.8 | — |
| CETETH-10 PHOSPHATE | 0.7 | — |
| DICETYL PHOSPHATE | 0.7 | — |
| Active ingredients | ≤1 | ≤1 |

The laboratory tests were conducted on “Latin Color Level TG3—Curl Type 4 (very curly)” locks from the supplier International Hair Importers & Products Inc. The treatment protocol used is as follows:
The locks of hair are rinsed under warm water (about 35° C.), washed with a deep-cleansing shampoo (Keratin Therapy Lisse Design Deep Cleansing Shampoo) and then photographed (pre-treatment evaluation).
2.5 g per lock of formulas A, B, C and E is applied. The product is distributed with a brush along the whole length of the lock, endeavouring to align the hair. The product is left to act for 20 minutes in a stove at 30° C. At the end of said time, the locks are 80-90% dried with a hairdryer.
The locks are straightened by passing a straightening iron set to 210° C. 10 times.
The locks are washed twice with a maintenance shampoo (Keratin Therapy Lisse Design Maintenance Shampoo) and then photographed (straightening evaluation at time zero, “t-zero”).
The locks are positioned in a stove at 75% relative humidity and 40° C. for 16 hours. At the end of said time, the locks are photographed (evaluation of degree of frizziness at time zero).
The combability evaluations are performed on dry hair and wet hair using a DIA-STRON MTT175 tester.
A 96-hour accelerated washing cycle is performed, setting to 45° C. a hermetically sealed container containing a solution of tapwater+4ml/L of maintenance shampoo (Keratin Therapy Lisse Design Maintenance Shampoo) wherein the locks are immersed. The locks are photographed at the end of the cycle.
The locks are positioned in a stove at 75% relative humidity and 40° C. for 16 hours. At the end of said time, the locks are photographed (evaluation of degree of frizziness after washing).
The images are acquired with a Sony α-5100 camera mounted on a specific structure able to keep the capture parameters constant. The images are processed with the Image J program (NIH) using the pre-set macros.
The data are analysed and processed with the Microsoft Office Excel program.

Evaluation of Degree of Frizziness

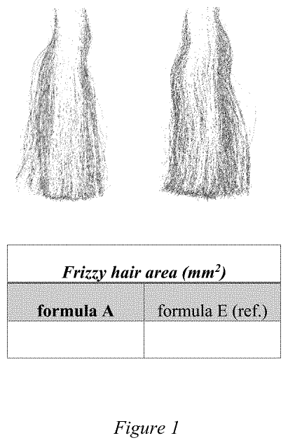

The degree of frizziness was evaluated at time zero (after straightening of the locks) and at the end of the accelerated washing cycles. The test protocol required the locks to be positioned in a stove at 75% relative humidity and 40° C. for 16 hours. At the end of said time the locks were photographed and processed with the Image J program. Using a pre-set macro the images are processed, only displaying the fibres responsible for the frizzy effect. The corresponding area is then quantified. FIG. 1 shows two representative images showing the photos after processing. The left-hand lock is representative of formula B, and the right-hand lock of reference formula E.

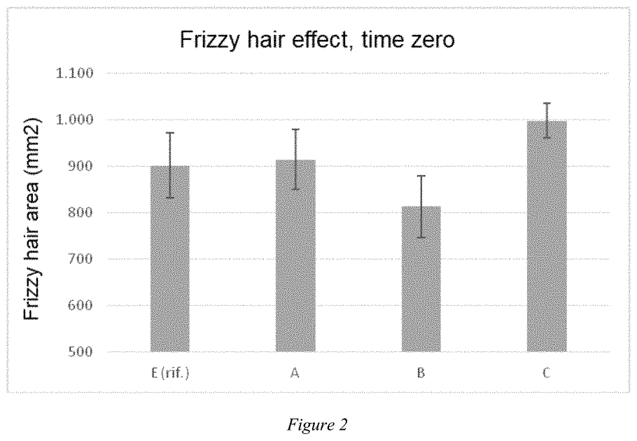

The data shown in Table 3 and FIG. 2 demonstrate that at time zero, formula B, containing 2.5% levulinic acid, improves frizziness by nearly 10% compared with reference formula E. However, no differences were observed between reference formula E and formula A containing 1% levulinic acid, and a worsening in frizziness was actually found in formula C containing 5% levulinic acid.

TABLE 3

| Formula | Frizzy hair area (mm²) | SD | % difference |
|---|---|---|---|
| E (ref.) | 902 | 70.0 | |
| A | 914 | 64.8 | 1.38 |
| B | 813 | 66.1 | −9.82 |
| C | 998 | 37.7 | 10.67 |

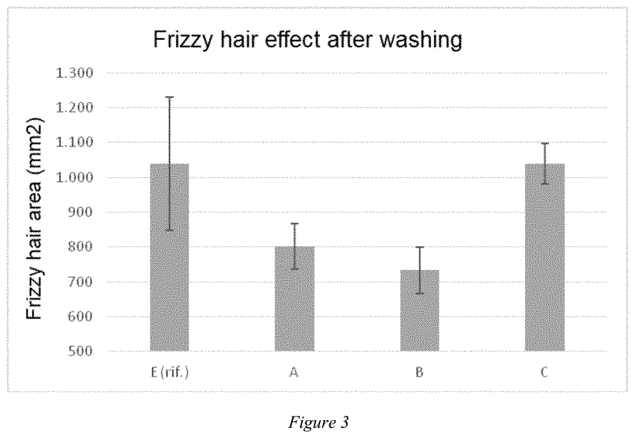

The data shown in Table 4 and FIG. 3 demonstrate that at the end of the accelerated washing cycle, formulas A and B, containing 1% and 2.5% levulinic acid respectively, exhibited a lower degree of frizziness than reference formula E. In particular, formula B exhibited a 29.43% improvement in the degree of frizziness compared with reference formula E. No differences were observed between the reference formula and formula C, containing 5% levulinic acid.

TABLE 4

| Formula | Frizzy hair area (mm²) | SD | % difference |
|---|---|---|---|
| E (ref.) | 1,039 | 191.8 | |
| A | 802 | 64.8 | −22.80 |
| B | 733 | 66.1 | −29.43 |
| C | 1039 | 59.0 | 0.03 |

Evaluation of Combability

Combability was evaluated on dry hair and wet hair at time zero (after straightening of the locks). The evaluations were performed with a DIA-STRON MTT175 tester. The locks are first analysed on dry hair, without handling. Friction when the comb (connected to the instrument) passes through the lock is measured. The measurements on wet hair are made by immersing the locks in tapwater at room temperature and removing the excess water before performing the measurement. The combability data are recorded by the software of the instrument, analysed, and expressed in Joules.

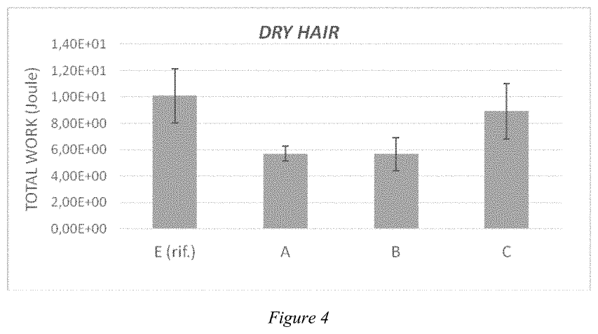

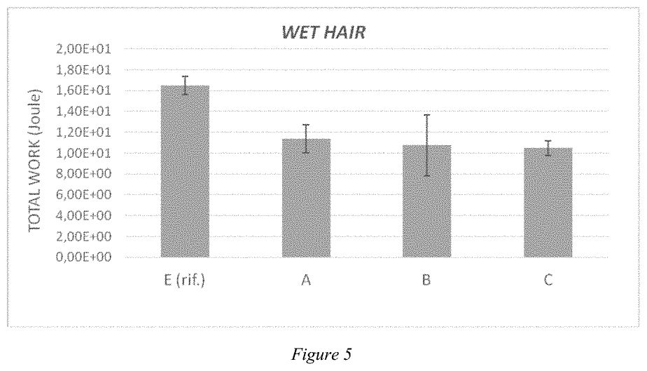

The data shown in Tables 5 and 6 and FIGS. 4 and 5 demonstrate that at the end of straightening of the locks, all the formulas containing levulinic acid (A, B, C) exhibited better combability on both dry and wet hair than reference formula E. The best results were obtained with formulas A and B, containing 1% and 2.5% of levulinic acid respectively. Compared with reference formula E, formula A exhibited 43.3% better combability on dry hair (Table 5 and FIGS. 4) and 30.9% on wet hair (Table 6 and FIG. 5). Formula B exhibited 43.7% better combability on dry hair (Table 5 and FIGS. 4) and 34.8% on wet hair (Table 6 and FIG. 5).

TABLE 5

| Formula | Total work (Joules) | SD | % difference |
|---|---|---|---|
| E (ref.) | 1.01E+01 | 2.04E+00 | |
| A | 5.71E+00 | 5.57E−01 | 43.3 |
| B | 5.68E+00 | 1.24E+00 | 43.7 |
| C | 8.92E+00 | 2.11E+00 | 11.5 |

TABLE 6

| Formula | Total work (Joules) | SD | % difference |
|---|---|---|---|
| E (ref.) | 1.65E+01 | 8.66E−01 | |
| A | 1.14E+01 | 1.34E+00 | 30.9 |
| B | 1.07E+01 | 2.91E+00 | 34.8 |
| C | 1.05E+01 | 7.13E−01 | 36.4 |

Evaluation of Straightening Efficacy

The straightening level was evaluated both at time zero and at the end of the accelerated washing cycle. This test simulates the various environmental factors that cause keratin fibre to regain its original shape in time. In practice, it simulates the return of the curl after 4 months' washing 3 times a week. The locks are immersed in a hermetically sealed container containing a solution of tapwater+4ml/L Keratin Therapy Lisse Design Maintenance Shampoo. The container is then placed in a stove at 45° C. for accelerated washing lasting 96 hours. The locks are photographed at the end of the cycle. The photos are then processed with the Image J program. The images are processed with a pre-set macro, and a grid is overlaid on each photo. Using the “multi-point” function of the program, a point is marked at each intersection between the columns of the grid and the lock.

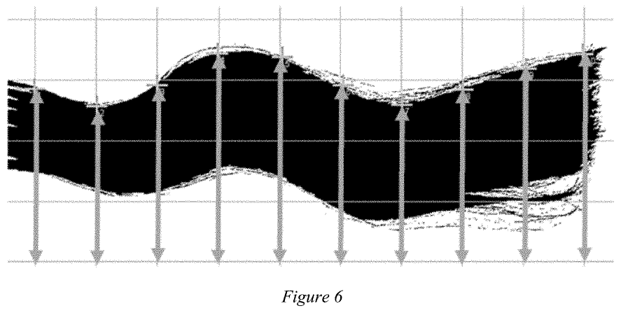

FIG. 6 shows a representative example of this processing. The data of each point on the y-axes are then acquired. The greater the curvature of the lock, the wider will be the distribution of said values. The mean value of said distances is then calculated, expressed in mm, and the data are then tabulated and processed with the Microsoft Office Excel program.

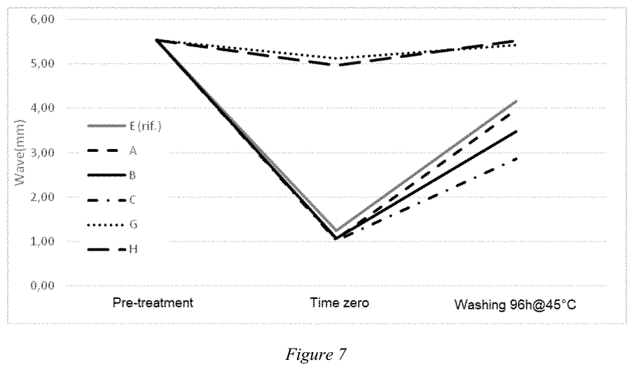

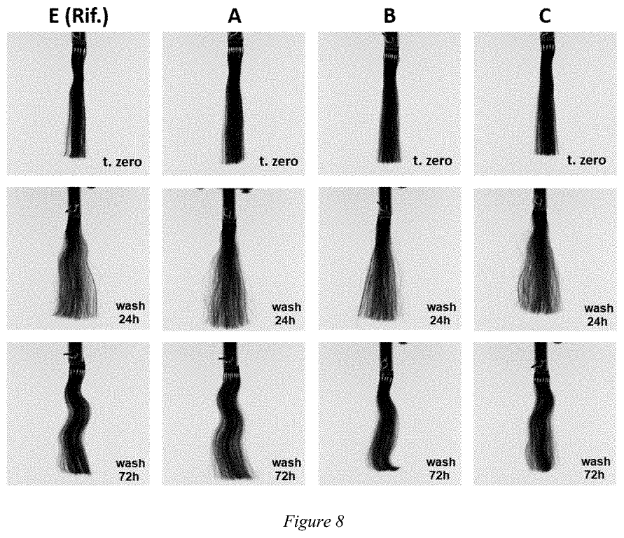

The data shown in Table 7 and FIG. 7 demonstrate that the straightening power of the formulas containing levulinic acid (A, B, C) is better than that of reference formula E. The improvement is observed at time zero, with an increase in straightening of 15.72%, 14.99% and 20.77% for formulas A, B, C respectively, and at the end of 96 hours' accelerated washing. In this case an increase in straightening of 4.70%, 16.41% and 31.26% is observed for formulas A, B, C respectively. FIG. 8 shows locks representative of each experimental group. It is clearly demonstrated that levulinic acid, added to a composition containing 15-20% by weight of glyoxylic acid, gives rise to an improvement in straightening even at time zero, and also better maintenance of the degree of straightness over time, with an effect proportional to the percentage content of levulinic acid. However, if the percentage of levulinic acid is increased at the expense of the glyoxylic acid content, as described in JP2019123701 (formulas G and H), straightening power is lost, to a level which is unacceptable for a semi-permanent professional treatment.

TABLE 7

| Pre-treatment | | Time zero | | | 96 h washing at 45° C. | | |
|---|---|---|---|---|---|---|---|
| | Wave | Wave | | | Wave | | |
| Formula | (mm) | (mm) | SD | % | (mm) | SD | % |
| E (ref.) | 5.53 | 1.25 | 0.43 | | 4.16 | 0.86 | |
| A | 5.53 | 1.05 | 0.25 | −15.72 | 3.96 | 0.64 | −4.70 |
| B | 5.53 | 1.06 | 0.21 | −14.99 | 3.48 | 0.85 | −16.41 |
| C | 5.53 | 1.03 | 0.23 | −20.77 | 2.86 | 0.74 | −31.26 |
| G | 5.53 | 5.12 | 1.17 | | 5.42 | 0.57 | |
| H | 5.53 | 4.97 | 0.49 | | 5.53 | 0.99 | |

In conclusion, the data demonstrate that levulinic acid improves the frizziness of hair, the maximum effect being provided by formula B, containing 2.5% levulinic acid. Better combability of the hair at the end of the straightening process is also demonstrated, with an effect not dependent on the percentage of levulinic acid. Finally, better straightening performance is demonstrated both at time zero and at the end of the accelerated washing period, with an effect proportional to the percentage of levulinic acid, provided that glyoxylic acid is present in the formulation in an amount ranging from 15 to 20% by weight, and the ratio between levulinic acid and glyoxylic acid ranges between 0.004 and 1.

Evaluation of Overall Performance With Panel Tests

Application tests were conducted on 5 models. The evaluations were conducted by 5 expert hairdressers, comparing formula B containing 2.5% levulinic acid used on half the head, and reference formula E on the other half. Formula B was selected because it is the one which offers the best compromise between straightening performance and improvement of frizziness. Both parameters are very important to define the overall performance of a straightening treatment. Various parameters were evaluated, as specified in Table 8. Each parameter was scored on a scale from 1 to 5, wherein 1 indicates “unsatisfactory”; 2 indicates “mediocre”; 3 indicates “fair”; 4 indicates “good” and 5 indicates “very good”. Application method no. 1 described above was used for the application. 5 tests were conducted, and the mean values are shown in Table 8. For the following parameters, improved values were found for formula B containing 2.5% levulinic acid, compared with reference formula E: ease of application and combability of hair during treatment; combability of hair at end of treatment; conditioning of hair at end of treatment; frizziness at end of treatment; degree of straightening at end of treatment; degree of straightening one month later. No deterioration was observed in other parameters, such as production of smoke during treatment, the shine of the hair and fading of the cosmetic colour at the end of the treatment, which proved comparable for the two formulas B and E. In particular, fading of the cosmetic colour was considered acceptable and easily corrected at the end of the straightening treatment. In conclusion, the application data confirm the findings obtained on locks.

TABLE 8

| | | Formula E |
|---|---|---|
| Parameter | Formula B | (ref.) |
| Ease of application and combability of | 4.2 | 3.4 |
| hair during treatment | | |
| Smoke during ironing stage | 3.2 | 3.2 |
| Colour fading of hair at end of treatment | 2.6 | 2.6 |
| Hair shine at end of treatment | 3.4 | 3.4 |
| Combability of hair at end of treatment | 4.2 | 3.2 |
| Hair conditioning at end of treatment | 4.6 | 4.0 |
| Frizziness at end of treatment | 4.6 | 3.8 |
| Degree of straightening at end of treatment | 4.6 | 4.2 |
| Degree of straightening one month later | 4.2 | 3.4 |

Evaluation of Formula in Emulsion Form

Application tests were conducted on 3 models. The evaluations were conducted by 5 expert hairdressers, comparing straightening formula D in emulsion form according to the invention on half the head with reference formula F containing a high percentage of fatty phase. Various parameters were evaluated, as specified in Table 9. Each parameter was scored on a scale from 1 to 5, wherein 1 indicates “unsatisfactory”; 2 indicates “mediocre”; 3 indicates “fair”; 4 indicates “good” and 5 indicates “very good”.

TABLE 9

| | | Formula F |
|---|---|---|
| Parameter | Formula D | (ref.) |
| Smoke during ironing stage | 2.2 | 1.0 |
| Combability of hair at end of treatment | 4.2 | 4.4 |
| Hair conditioning at end of treatment | 4.4 | 4.6 |
| Frizziness at end of treatment | 4.6 | 4.6 |
| Degree of straightening at end of treatment | 4.6 | 4.6 |

Both formulas make more smoke during the ironing stage than formulas B and E with an aqueous base, but Formula F, as expected, makes much more.

As stated, the fatty phase contained in the straightening compositions in emulsion form improves the cosmetic properties of the hair at the end of the treatment, but gives rise to the formation of a considerable amount of smoke during the passage of the hot iron. To obviate this problem, said compositions are usually rinsed before ironing, with a resulting loss of straightening performance. The data obtained with the application tests indicate that formula B according to the present invention, developed in liquid form and containing 2.5% levulinic acid, guarantees excellent cosmetic properties, similarly to the formulas containing a fatty phase. As expected, it makes much less smoke during the ironing stage than the formulas containing a fatty phase, and the straightening performance is excellent. Compared with reference formula E, formula B according to the present invention produces a definite improvement in cosmetic properties and straightening performance.

1. Semi-permanent hair straightening compositions in liquid form with an aqueous base, or in emulsion form with a fatty phase, comprising 15 to 20% by weight of glyoxylic acid and 0.1 to 5% by weight of levulinic acid.

2. Compositions according to claim 1 comprising 2% to 3% by weight of levulinic acid.

3. Compositions according to claim 1, wherein the ratio of levulinic acid to glyoxylic acid ranges from 0.004 to 1.

4. Compositions according to claim 3 wherein the ratio of levulinic acid to glyoxylic acid ranges from 0.1 to 0.2.

5. Compositions according to claim 1, having a pH ranging from 0.5 to 3.0.

6. Compositions according to claim 1, further comprising non-ionic, amphoteric, and cationic surfactants.

7. Compositions according to claim 1, further comprising pyruvic acid.

9. Compositions according to claim 1, further comprising solvents, fats, polymers, oligosaccharides and modified oligosaccharides, carbohydrates and derivatives, glycerols, polyols, triglycerides, hydrocarbons, lanolin, opacifiers, silicones, protein hydrolysates, amino acids, complexing agents, UV filters, pigments, preservatives and conditioning fragrances, dyes.

10. A semi-permanent hair straightening method comprising
applying the compositions of claim 1 to the hair and subsequent heat treatment,
washing and rinsing.
